(19) United States
(12) Patent Application Publication
(10) Pub. No.: US 2026/0263105 A1
(43) Pub. Date: Sep. 10, 2026

(54) TEMPERATURE CONTROL IN ULTRASONIC SURGICAL INSTRUMENTS

(71) Applicant: CILAG GMBH INTERNATIONAL, Zug (CH)

(72) Inventors: Nicholas Alexander MCDONOUGH, West Harrison, IN (US); Guion Yuvano LUCAS, Cincinnati, OH (US); Patrick Jarvis SCOGGINS, Loveland, OH (US); Craig Nelson FALLER, Batavia, OH (US)

(73) Assignee: CILAG GMBH INTERNATIONAL, Zug (CH)

(21) Appl. No.: 19/554,181

(22) Filed: Mar. 2, 2026

Related U.S. Application Data
(60) Provisional application No. 63/769,483, filed on Mar. 10, 2025.
(60) Provisional application No. 63/770,046, filed on Mar. 11, 2025.

(51) Int. Cl. A61B 17/32; A61B 17/00

(52) U.S. Cl. CPC A61B 17/320068; A61B 2017/00084; A61B 2017/00137; A61B 2017/00199; A61B 2017/320095

(57) ABSTRACT

A surgical system is disclosed that includes a surgical instrument comprising an ultrasonic drive system that includes an ultrasonic blade and an ultrasonic transducer, a temperature sensor to sense a temperature of the ultrasonic transducer, and a controller operable to, receive a user input, set a temperature range of the ultrasonic blade based on the user input, drive, with the ultrasonic transducer, the ultrasonic blade in a first manner, sense, with the temperature sensor, the temperature of the ultrasonic transducer, determine the natural frequency of the ultrasonic drive system, determine a temperature of the ultrasonic blade based on the natural frequency of the ultrasonic drive system and the temperature of the ultrasonic transducer, compare the temperature of the ultrasonic blade to the temperature range, and drive, with the ultrasonic transducer, the ultrasonic blade a second manner different than the first manner based on the comparison.

### CROSS-REFERENCE TO RELATED APPLICATION

The present application claims priority under 35 U.S.C. § 119(e) to U.S. Provisional Application No. 63/769,483, titled “PRESSURE CONTROL IN ULTRASONIC SURGICAL INSTRUMENTS”, filed Mar. 10, 2025, the disclosure of which is hereby incorporated by reference in its entirety.

The present application claims priority under 35 U.S.C. § 119(e) to U.S. Provisional Application No. 63/770,046, titled “TEMPERATURE CONTROL IN ULTRASONIC SURGICAL INSTRUMENTS”, filed Mar. 11, 2025, the disclosure of which is hereby incorporated by reference in its entirety.

### BACKGROUND

The present disclosure relates to surgical instruments and, more particularly, to ultrasonic surgical instruments that are configured to cut and seal tissue.

Ultrasonic surgical instruments have become widely adopted in modern surgery due to their ability to simultaneously cut and seal tissue with high precision. These devices operate by converting high-frequency electrical energy into mechanical vibrations, which are then applied to an ultrasonic blade. The resulting ultrasonic waves (oscillations) create localized heating through friction, allowing the instrument to seal and/or cut blood vessels or tissues.

While ultrasonic surgical instruments have proven highly effective in many procedures, there is an inherent challenge that arises during their use. Surgeons often rely on the ultrasonic device to achieve precise tissue sealing, but in certain situations, they may inadvertently cut the blood vessels or tissue. This issue can occur because of the high energy levels involved in ultrasonic cutting and sealing, as well as the difficulty in maintaining a consistent pressure, temperature, and energy output during complex procedures.

Current ultrasonic surgical instruments typically rely on manual control by the surgeon to differentiate between cutting and sealing actions. However, the fine line between these two operations is often difficult to manage with precision, especially in high-stress or time-sensitive surgical environments. Surgeons may struggle to adjust the intensity, frequency, or amplitude of the ultrasonic energy quickly enough to maintain the intended balance between cutting and sealing.

Accordingly, systems and methods for controlling whether an ultrasonic surgical instrument cuts or seals tissue are desired.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 illustrates an example surgical system that includes a generator usable with various surgical instruments, in accordance with at least one aspect of the present disclosure.

FIG. 2 is a schematic block diagram of the surgical system of FIG. 1, in accordance with at least one aspect of the present disclosure.

FIG. 3 is a schematic flow diagram for controlling temperature of an ultrasonic blade of an ultrasonic instrument, according to at least one aspect of the present disclosure.

FIG. 4 is a schematic flow diagram for controlling pressure applied to tissue by a clamp arm of an ultrasonic instrument, according to at least one aspect of the present disclosure.

### DETAILED DESCRIPTION

Applicant of the present application owns the following U.S. Patent Application filed concurrently herewith, the disclosure of which is hereby incorporated by reference in its entirety herein:

U.S. patent application Ser. No. 19/554,185, filed Mar. 2, 2026, titled PRESSURE CONTROL IN ULTRASONIC SURGICAL INSTRUMENTS.

The present disclosure relates to surgical instruments and, more particularly, to ultrasonic surgical instruments that are configured to cut and seal tissue.

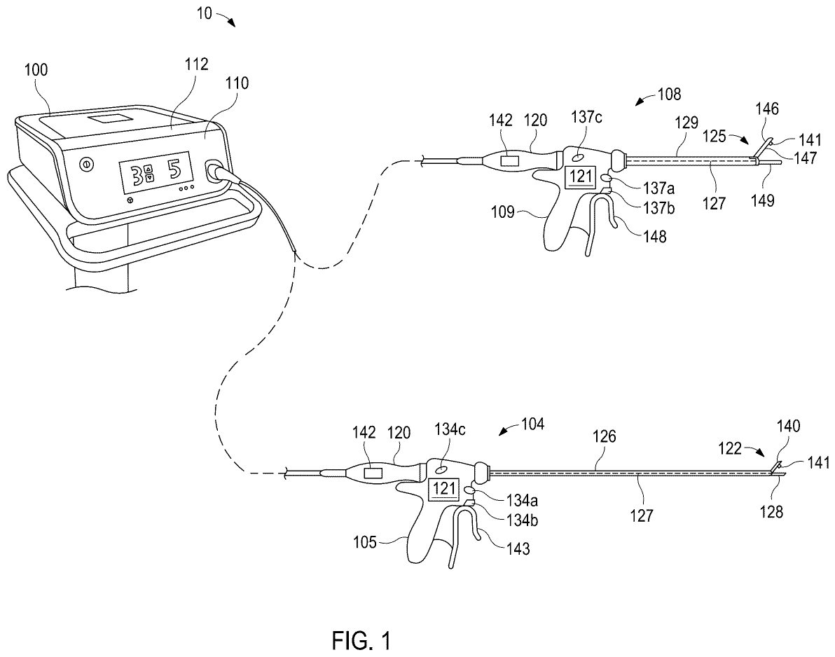

FIG. 1 illustrates an example surgical system 10 comprising a generator 100 usable with various surgical instruments 104, 108, in accordance with at least one aspect of the present disclosure. The first surgical instrument 104 is an ultrasonic surgical instrument 104 that includes a handpiece or “housing” 105, an ultrasonic transducer 120, a shaft 126 extending from the housing 105, and an end effector 122 arranged at a distal end of the shaft 126.

The end effector 122 includes an ultrasonic blade 128 operably coupled to the ultrasonic transducer 120 via a waveguide 127 (shown in phantom) that extends through the shaft 126 and a clamp arm 140. The ultrasonic transducer 120, the waveguide 127, and the ultrasonic blade 128 may form at least part of an ultrasonic drive system 111. The handpiece 105 comprises a motor 121 operable to move (rotate) the clamp arm 140 relative to the ultrasonic blade 128, a trigger 143 to manually position the clamp arm 140 and/or to actuate (energize) the motor 121, and a combination of toggle buttons 134a, 134b, 134c to energize and drive the ultrasonic blade 128 or other functions. The toggle buttons 134a-c can be configured to energize the ultrasonic transducer 120 with the generator 100.

The clamp arm 140 may include a first or “gap” sensor 141, such as a Hall-Effect sensor, to sense a gap (distance) between the clamp arm 140 and the ultrasonic blade 128. The ultrasonic surgical instrument 104 may further include a second or “temperature” sensor 142 for sensing a temperature of the ultrasonic transducer 120. In some embodiments, the temperature sensor 142 may be coupled to a housing of the ultrasonic transducer 120.

The second surgical instrument 108 is a multifunction surgical instrument 108 that includes a handpiece 109 or “housing”, a shaft 129 extending from the housing 109, and an end effector 125 arranged at the distal end of the shaft 129. The end effector 125 includes an ultrasonic blade 149 and a clamp arm 146 pivotable toward and away from the ultrasonic blade 149. The ultrasonic blade 149 is operably coupled to the ultrasonic transducer 120 via a waveguide 127 (shown in phantom) that extends through the shaft 129. The ultrasonic transducer 120, the waveguide 127, and the ultrasonic blade 149 may form at least part of an ultrasonic drive system 111. The clamp arm 146 includes one or more electrodes 147 coupled to a pole of the generator 100 (e.g., a positive pole). The ultrasonic blade 149 may form the second pole (e.g., the negative pole) and may also be coupled to the generator 100.

The handpiece 109 includes a motor 121 to move (rotate) the clamp arm 146 relative to the ultrasonic blade 149, a trigger 148 to manually position the clamp arm 140 and/or to actuate (energize) the motor 121, and a combination of toggle buttons 137a, 137b, 137c to energize and drive the ultrasonic blade 149, energize the electrodes, or other functions. The toggle buttons 137a-c can energize the ultrasonic transducer 120 with the generator 100 or energize the electrodes 147 with a bipolar energy source contained within the generator 100. For example, RF energy may be applied to the electrode(s) 147 in the clamp arm 146, through the tissue located between the clamp arm 146 and the ultrasonic blade 149, and through the ultrasonic blade 149 back to the generator 100.

The clamp arm 140 may include a first or “gap” sensor 141, such as a Hall-Effect sensor, to sense a gap (distance) between the clamp arm 146 and the ultrasonic blade 149. The multifunction surgical instrument 108 may further include a second or “temperature” sensor 142 for sensing a temperature of the ultrasonic transducer 120. The temperature sensor 142 may be coupled to a housing of the ultrasonic transducer 120. Considering the multifunction surgical instrument 108 is configured to deliver ultrasonic energy and RF energy, the multifunction surgical instrument 108 may alternatively be referred to as an ultrasonic surgical instrument or an RF surgical instrument.

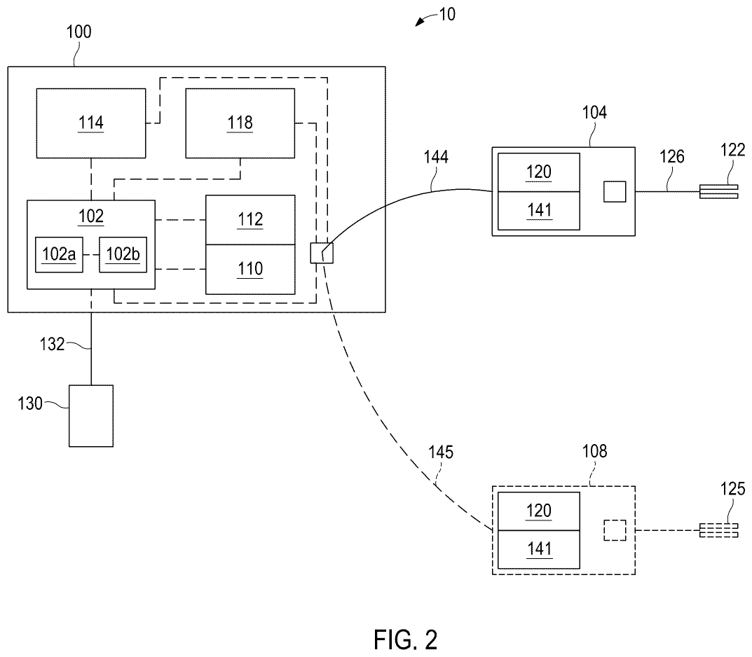

FIG. 2 is a schematic block diagram of the surgical system 10 of FIG. 1, in accordance with at least one aspect of the present disclosure. The generator 100 may comprise several separate functional elements, such as modules and/or blocks. Different functional elements or modules may be configured for driving the surgical instruments 104, 108. For instance, the generator 100 may include an ultrasonic drive circuit or “ultrasonic generator” 114 to drive the ultrasonic surgical instrument 104 via a cable 144 and a combined RF/ultrasonic drive circuit or “RF/ultrasonic generator” 118 to drive the multifunction surgical instrument 108 via a cable 145. Considering the combined RF/ultrasonic drive circuit 118 is configured to deliver ultrasonic energy and RF energy, the combined RF/ultrasonic drive circuit 118 may alternatively be referred to as an ultrasonic drive circuit or “ultrasonic generator” or an RF drive circuit or “RF generator”.

The ultrasonic drive circuit 114 and the combined RF/ultrasonic drive circuit 118 may produce a drive signal or signals of particular voltages, currents, and frequencies, e.g., 55,500 cycles per second (Hz). The drive signal or signals may be provided to the surgical instruments 104, 108, respectively, and more specifically, to the ultrasonic transducer 120. The generator 100 may include a controller 102 that is in operable communication with the ultrasonic drive circuit 114 and the combined RF/ultrasonic drive circuit 118 and may be operable to control the same to deliver the drive signal(s) such that the ultrasonic drive systems 111 (e.g. the ultrasonic transducer 120, the waveguide 127, and the ultrasonic blades 128, 149) are driven at their natural frequencies fₙ. The controller 102 may continuously, or periodically, adjust the drive signal(s) to maintain the ultrasonic drive system 111 at their natural frequency fₙ. The generator 100 may be configured to produce a drive signal of a particular voltage, current, and/or frequency output signal that can be stepped or otherwise modified with high resolution, accuracy, and repeatability.

The generator 100 may be activated to provide the drive signal to the ultrasonic transducer 120 in any suitable manner. For example, the generator 100 may be in communication with a foot switch 130 via a foot switch cable 132. A clinician may activate the ultrasonic transducer 120 by depressing the foot switch 130. In addition, or instead of the foot switch 130, the ultrasonic surgical instrument 104 may utilize the toggle buttons 134a-c, 137a-c (FIG. 1) positioned on the handpiece 105, 109 that, when activated, may cause the generator 100 to activate the ultrasonic transducer 120. The toggle buttons 134a-c, 137a-c may be used to determine an operating mode of the surgical instruments 104, 108. For example, when the first toggle button 134a, 137a is depressed, the generator 100 may provide a first drive signal to the ultrasonic transducer 120, causing it to produce a first ultrasonic energy output. Depressing the second toggle button 134b, 137b may cause the generator 100 to provide a second drive signal to the ultrasonic transducer 120 that is different than the first drive signal, causing the generator 100 to produce a second ultrasonic energy output that is different (less than or greater than) from the first ultrasonic energy output.

The generator 100 may further include an input device 110 located on a front panel of the generator 100 console. The input device 110 may comprise any suitable device that generates signals that can be used by the generator 100 (e.g., by the controller 102 contained in the generator 100) to control the operation of the generator 100 (e.g., operation of the ultrasonic drive circuit 114, the combined RF/ultrasonic drive circuit 118, or the motors 121, or combinations thereof). The input device 110 may include one or more of buttons, switches, thumbwheels, keyboard, keypad, touch screen monitor, pointing device, remote connection to a general purpose or dedicated computer, or any combination thereof. The input device 110 may comprise a suitable user interface, such as one or more user interface screens displayed on a touch screen monitor. Accordingly, by way of the input device 110, the user can set or program various operating parameters of the generator 100, such as, for example, current (I), voltage (V), frequency (f), and/or period (T) of a drive signal or signals generated by the ultrasonic drive circuit 114 and/or the combined RF/ultrasonic drive circuit 118.

The generator 100 may also include one or more output devices 112, such as an output indicator, located, for example, on a front panel of the generator 100. The output device 112 can include one or more devices for providing a sensory feedback to a user. Such devices may comprise visual feedback devices (e.g., incandescent lamps, LEDs, graphical user interface, display, analog indicator, digital indicator, bar graph display, digital alphanumeric display, liquid crystal display (LCD) screen, light emitting diode (LED) indicators), audio feedback devices (e.g., a speaker, buzzer, audible, computer generated tone, computerized speech, voice user interface (VUI) to interact with computers through a voice/speech platform), or tactile feedback devices (e.g., any type of vibratory feedback, haptic actuator).

The controller 102 may include a processor 102a and a memory 102b comprising storage media readable by the processor 102a. The memory 102b may store software or software instructions executable by the processor 102a. Based on instructions provided by the software, the controller 102 may be configured to control various aspects of the system 10, such as the output of the output device 112, a position of the clamp arms 140, 146 via the motors 121, or a voltage, current, and frequency provided to the ultrasonic transducers 120 via the drive circuits 114, 118, or combinations thereof.

### Temperature Control in Ultrasonic Surgical Instruments

Current ultrasonic surgical instruments typically rely on manual control by the surgeon to differentiate between cutting and sealing actions while treating tissue. However, the fine line between these two operations is often difficult to manage with precision, and surgeons who intend to merely seal a blood vessel or tissue may inadvertently cut the same. Accordingly, systems and methods for controlling whether the ultrasonic blade cuts or seals tissue are desired.

The inventors have found that tissue is typically sealed, but not cut, when the temperature of the ultrasonic blade is within a range of about 140° C. to 160° C., and more preferably, at about 150° C. Temperatures that exceed this temperature range tend to cause the tissue to be cut as opposed to merely sealed. Accordingly, systems and methods for controlling the temperature of the ultrasonic blade to control whether the ultrasonic blade cuts or seals tissue are desired.

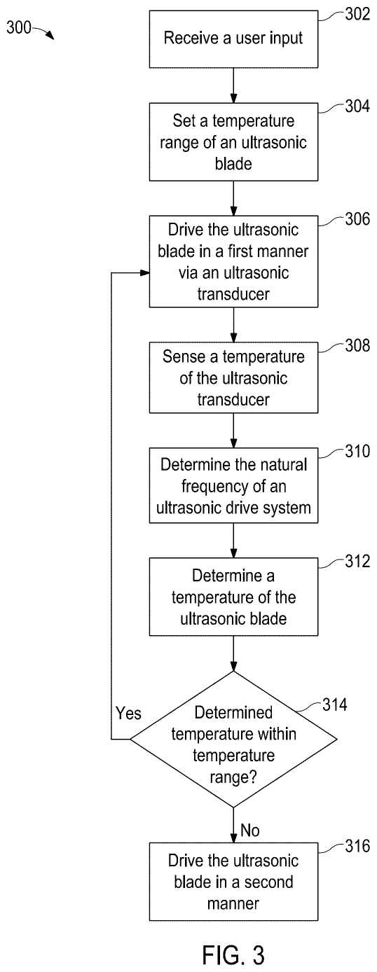

FIG. 3 is a schematic flow diagram of an example method 300 for controlling the temperature of an ultrasonic blade of an ultrasonic instrument, according to at least one aspect of the present disclosure. The method 300 may be embodied as an algorithm, stored in the memory 102b (FIG. 2) of the controller 102 (FIG. 2) and may be executed by the processor 102 (FIG. 2) of the controller 102, such as based on an input provided to the controller 102 by a user at the input interface 110 (FIG. 2).

With reference to FIGS. 1 and 3, the method 300 may include receiving a user input, as at step 302. For instance, a user may desire to treat tissue with one of the surgical instruments 104, 108. Accordingly, the user may provide an input to controller 102 (FIG. 2), such as via the input device 110 or via one or more of the toggle buttons 134a-c, 137a-c positioned on the handpieces 105, 109, or a combination thereof. The input may be indicative of a desired operating mode of the surgical instruments 104, 108. The operating modes may include a first or “tissue sealing” mode, in which the surgical instruments 104, 108 are controlled by the controller 102 to seal, but not cut, tissue grasped between the clamp arms 140, 146 and the ultrasonic blades 128, 149, or a second or “tissue cutting” mode, in which the surgical instruments 104, 108 are controlled by the controller 102 to cut tissue grasped between the clamp arms 140, 146 and the ultrasonic blades 128, 149. The controller 102 may receive an input indicative of the first mode based on the user interacting with the first toggle button 134a or an input indicative of the second mode based on the user interacting with the second toggle buttons 134b.

The method 300 further includes setting a temperature range of an ultrasonic blade, as at step 304. For instance, based on the user input, the controller 102 may retrieve a temperature range associated with the desired operating mode of the surgical instruments 104, 108. The temperature ranges may be stored in the memory 102b of the controller 102 or may be provided to the controller 102 by the user, such as via the input interface 110. The temperature range of the first operating mode may include a first minimum value and a first maximum value, and the temperature range of the second operating mode may include a second minimum value different (less or greater) than the first minimum value, and a second maximum value different (less or greater) than the first maximum value. The first minimum and first maximum values may be about 145° C. and about 155° C., respectively, and the second minimum and second maximum values may be about 270° C. and about 280° C., respectively. In other embodiments, the first minimum value may be less than 145° C. (e.g., about 130° C., 135° C., or 140° C.) or greater than 145° C. (e.g., about 150° C., 155° C., 160° C.). In other embodiments, the first maximum value may be less than 155° C. (e.g., about 140° C., 145° C., or 150° C.) or greater than 155° C. (e.g., about 160° C., 165° C., 170° C.). In other embodiments, the second minimum value may be less than 270° C. (e.g., about 255° C., 260° C., or 265° C.) or greater than 270° C. (e.g., about 275° C., 280° C., 285° C.). In other embodiments, the second maximum value may be less than 280° C. (e.g., about 265° C., 270° C., or 275° C.) or greater than 280° C. (e.g., about 285° C., 290° C., 295° C.).

Alternatively, the method 300 may include setting a temperature set point associated with the desired operating mode of the surgical instruments 104, 108. The temperature set point of the first operating mode may be a first value and the temperature set point of the second operating mode may be a second value different (greater or less) than the first value. In some embodiments, the first and second values may be about 150° C. and about 275° C., respectively. In other embodiments, the first value may be less than 150° C. (e.g., about 135° C., 140° C., or 145° C.) or greater than 150° C. (e.g., about 155° C., 160° C., or 165° C.). In other embodiments, the second value may be less than 275° C. (e.g., about 260° C., 265° C., or 270° C.) or greater than 275° C. (e.g., about 280° C., 285° C., or 290° C.).

The method 300 further includes driving the ultrasonic blade in a first manner via an ultrasonic transducer, as at step 306. For instance, after setting the temperature range of the ultrasonic blade (step 304), the controller 102 may cause the ultrasonic drive circuit 114 or the combined RF/ultrasonic drive circuit 118 to provide a first drive signal to the ultrasonic transducer 120, thereby causing the ultrasonic transducer 120 to oscillate (drive) the ultrasonic blade 128 at the natural frequency fₙ of the ultrasonic drive system 111. The first drive signal may cause the ultrasonic blade 128 to vibrate in a first manner, which may be the ultrasonic blade 128 being moved (vibrated) within a first range of motion d₁ or moved (vibrated) at a first frequency f₁.

The controller 102 may cause the ultrasonic drive circuit 114 or the combined RF/ultrasonic drive circuit 118 to provide the first drive signal to the ultrasonic transducer 120 based on the user providing an input to the controller 102, such as via the input device 110, the footswitch 130 (FIG. 2), or the toggle buttons 134a-c, 137a-c. In other embodiments, the controller 102 may cause the ultrasonic drive circuit 114 or the combined RF/ultrasonic drive circuit 118 to provide the first drive signal to the ultrasonic transducer 120 automatically, such as based on the controller 102 detecting the tissue being captured between the clamp arm 140, 146 and the ultrasonic blade 128, 149. The controller 102 may detect the tissue being captured by applying a subtherapeutic energy signal to the electrode 147 and sensing (detecting) a change in impedance. The controller 102 may also detect the tissue being captured using a force sensor (not shown) coupled to the ultrasonic drive system 111. For instance, the force sensor may be a strain gauge coupled to the waveguide 127 and the controller 102 may detect the tissue being captured based on data from the strain gauge, such as a change in sensed force when the waveguide 127 is bent (deflected) when capturing tissue between the clamp arm and ultrasonic blade. In other embodiments, the force sensor may be coupled to the clamp arm 140 or the ultrasonic blade to directly measure force applied to the tissue, and the controller 102 may detect the tissue being captured based on the measured force.

The method 300 further includes sensing a temperature of the ultrasonic transducer, as at step 308. For instance, as the controller 102 drives the ultrasonic blade 128, as at step 306, the controller 102 may sense, via the sensor 142, the temperature of the ultrasonic transducer 120. The controller 102 may sense the temperature continuously or periodically.

The method 300 further includes determining the natural frequency of an ultrasonic drive system, as at step 310. As discussed elsewhere herein, the ultrasonic transducer 120, the ultrasonic blade 128, and the waveguide 127 extending therebetween form at least part of the ultrasonic drive system 111 of the ultrasonic instrument 104. While driving the ultrasonic blade 128 (step 306), the controller 102 may monitor and periodically (or continuously) adjust the first drive signal to maintain the ultrasonic drive system 111 being driven at its natural frequency fₙ. The natural frequency fₙ of the ultrasonic drive system 111 is based on, among other things, the temperature of the ultrasonic drive system 111, which may change (fluctuate) as the ultrasonic blade 128 vibrates against tissue. For example, due to friction between the ultrasonic blade and the tissue, the temperature of the ultrasonic drive system 111 may change (increase) over time. Accordingly, the controller 102 may periodically, or continuously, determine the natural frequency fₙ of the ultrasonic drive system by monitoring for phase shift of the first drive signal. Based on a detected phase shift, the controller 102 may adjust the first drive signal to maintain the ultrasonic blade vibrating at its natural frequency fₙ.

The method 300 further includes determining a temperature of the ultrasonic blade, as at step 312. For instance, based on the sensed temperature of the ultrasonic transducer 120 and the determined natural frequency of the ultrasonic drive system 111, the controller 102 can determine (estimate) the temperature of the ultrasonic blade 128 according to the following equation:

$${T}_{blade} = {m}_{1} * {f}_{n} + {b}_{1} - ( {m}_{2} * {T}_{transducer} + {b}_{2} )$$

where T_{blade} is the temperature of the ultrasonic blade 128, T_{transducer} is the temperature of the ultrasonic transducer 120 (as sensed by sensor 142; step 308), fₙ is the natural frequency of the ultrasonic drive system (as determined by the controller 102; step 310), m₁ and b₁ are constants associated with the ultrasonic blade 128 determined experimentally, and m₂, b₂ are constants associated with the ultrasonic transducer 120 determined experimentally. More specifically, m₁, b₁, m₂ and b₂ can be obtained in experiments by collecting empirical data of the temperature of the ultrasonic blade 128 using an infrared camera while collecting the corresponding natural frequency fₙ and temperature of the transducer T_{transducer}. Linear approximations are made to establish the relationship between T_{blade}, natural frequency fₙ, and the temperature of the transducer 120 T_{transducer}, thus yielding m₁, b₁, m₂ and b₂.

The method 300 further includes comparing the determined temperature of the ultrasonic blade to the temperature range, as at step 314. For instance, when the user selects the first mode at step 302, the controller 102 may compare the determined temperature to the temperature range associate with the first mode (e.g. about 145° C. to about 155° C.). Similarly, when the user selects the second mode at step 302, the controller 102 may compare the determined temperature to the temperature range associate with the second mode (e.g. about 270° C. to about 280° C.). Alternatively, in instances where a temperature set point was set at step 304, the method 300 includes comparing the determined temperature of the ultrasonic blade to the temperature set point.

If the determined temperature is within the temperature range (or at the temperature set point), the method 300 may proceed back to step 306 and continue driving the ultrasonic blade 128 in the first manner. If the determined temperature is outside the temperature range (or different than the temperature set point), however, the method 300 may proceed to step 316, which includes driving the ultrasonic blade in a second manner different than the first manner.

In some applications, the second manner may consist of moving (vibrating) the ultrasonic blade 128 within a second range of motion d₂ different than (less than or greater than) the first range of motion d₁, but at the same frequency (e.g., the first frequency f₁). In other applications, the second manner may consist of moving (vibrating) the ultrasonic blade 128 at a second frequency f₂ different than (less than or greater than) the first frequency f₁, but within the same range of motion (e.g., the first range of motion d₁). In other applications, the second manner may consist of moving (vibrating) the ultrasonic blade 128 at a second frequency f₂ different than (less than or greater than) the first frequency f₁ and within a second range of motion d₂ different than (less than or greater than) the first range of motion d₁.

For example, the controller 102 may determine, at step 314, that the temperature of the ultrasonic blade 128 is greater than the maximum value (or set point value) for the associated operating mode. Accordingly, in an effort to reduce the temperature of the ultrasonic blade 128, the controller 102 may adjust the first drive signal to a second drive signal different than the first drive signal to change (reduce) the range of motion and/or frequency of the ultrasonic blade 128 in an effort to decrease the temperature of the ultrasonic blade 128 to below the maximum value (or to the set point value). As another example, the controller 102 may determine that the temperature of the ultrasonic blade 128 is less than the minimum value (or set point value) for the associated operating mode. In such a scenario, the controller 102 may adjust the first drive signal to a third drive signal different than the first or second drive signals to change (increase) the range of motion and/or frequency of the ultrasonic blade 128 in an effort to increase the temperature of the ultrasonic blade 128 to above the minimum value (or to the set point value).

Accordingly, the foregoing systems and methods function to maintain the temperature of the ultrasonic blade within a specific range (or at a particular value) that is conducive to sealing or cutting of the tissue. This enables a user to selectively control whether an ultrasonic surgical instrument, such as the ultrasonic surgical instrument 104 or the multifunction surgical instrument 108, cuts or seals tissue.

### Pressure Control in Ultrasonic Surgical Instruments

Current ultrasonic surgical instruments, such as the ultrasonic surgical instrument 104 and the multifunction surgical instrument 108, typically rely on manual control by the surgeon to differentiate between cutting and sealing actions while treating tissue. However, the fine line between these two operations is often difficult to manage with precision, and surgeons who intend to merely seal a blood vessel or tissue may inadvertently cut the same. Accordingly, systems and methods for controlling whether the ultrasonic blade cuts or seals tissue are desired.

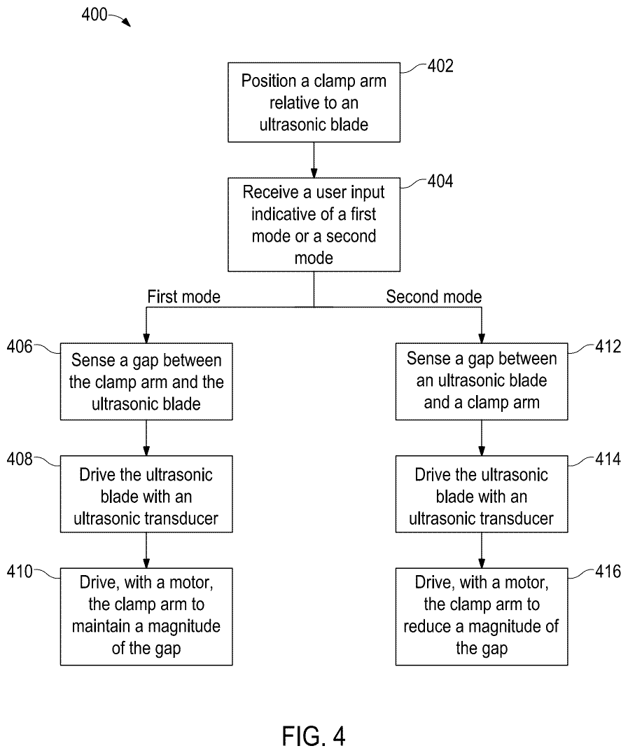

FIG. 4 is a schematic flow diagram of an example method 400 for controlling pressure applied to tissue by a clamp arm of an ultrasonic surgical instrument, according to at least one aspect of the present disclosure. The method 400 may be embodied as an algorithm, stored in the memory 102b (FIG. 2) of the controller 102 (FIG. 2) and may be executed by the processor 102a (FIG. 2) of the controller 102, such as based on an input provided to the controller 102 by a user at the input interface 110 (FIG. 2).

With reference to FIGS. 1 and 4, the method 400 may include positioning a clamp arm relative to an ultrasonic blade, as at step 402. For instance, a user may desire to operate on tissue with one of the surgical instruments 104, 108. Accordingly, the user may navigate the end effector 122 of the surgical instrument 104 to the tissue and position the tissue between the clamp arm 140 and the ultrasonic blade 128. The user may then manually position the clamp arm 140 relative to the ultrasonic blade 128 using the trigger 143 to capture tissue therebetween. For instance, the user may rotate the trigger 143 toward the housing 105, thereby causing motion of the clamp arm 140 via a closure system (e.g., links, shafts, gears, etc.; not shown) arranged within the surgical instrument 104. Alternatively, or in combination therewith, the user may position the clamp arm 140 relative to the ultrasonic blade 128 using the motor 121. For instance, the user may provide an input to the input device 110 or the ultrasonic instrument 104, such as via the trigger 143, which may cause the controller 102 to actuate the motor 121 to rotate (position) the clamp arm 140 relative to the ultrasonic blade 128 to capture tissue therebetween. A gap may be defined the clamp arm 140 and the ultrasonic blade 128 rotating the clamp arm 140 toward the ultrasonic blade 128 may reduce (shrink) the gap.

The method 400 further includes receiving a user input, as at step 404. The user input may be indicative of a desired mode of the surgical instrument 104, 108. The mode may include a first or “tissue sealing” mode, in which the surgical instruments 104, 108 are controlled by the controller 102 to seal, but not cut, the tissue grasped between the clamp arms 140, 146 and the ultrasonic blades 128, 149, or a second or “tissue cutting” mode, in which the surgical instruments 104, 108 are controlled by the controller 102 to cut the tissue grasped between the clamp arms 140, 146 and the ultrasonic blades 128, 149. The controller 102 may receive the user input via the input device 110 or via one or more of the toggle buttons 134a-c, 137a-c positioned on the handpieces 105, 109, or a combination thereof. For instance, the controller 102 may receive an input indicative of the first mode based on the user interacting with (pressing) the first toggle button 134a or an input indicative of the second mode based on the user interacting with (pressing) the second toggle buttons 134b.

When the user input corresponds to the first (tissue sealing) mode, the method 400 may include sensing a gap between the ultrasonic blade and the clamp arm, as at step 406. For instance, based on the controller 102 receiving a user input indicative of the first (tissue sealing) mode of the surgical instrument 104, the controller 102 may sense, via the sensor 141, the gap (distance) between the clamp arm 140 and the ultrasonic blade 128.

The method 400 may further include driving the ultrasonic blade with an ultrasonic transducer, as at step 408. For instance, the controller 102 may energize (actuate) the ultrasonic transducer 120, such as with the ultrasonic generator 114 (FIG. 2), to oscillate (drive) the ultrasonic blade 128. The controller 102 may energize the ultrasonic transducer 120 based on a user providing an input to the controller 102, such as via the input device 110 or the footswitch 130 (FIG. 2), or based on a user providing an input to the instrument 104, such as via the toggle buttons 134a-c. In other embodiments, the controller 102 may energize (actuate) the ultrasonic transducer 120 automatically, such as based on the controller 102 detecting the tissue being captured between the clamp arm 140, 146 and the ultrasonic blade 128, 149. The controller 102 may detect the tissue being captured by applying a subtherapeutic energy signal to the electrode 147 and sensing (detecting) a change in impedance.

The method 400 may further include driving, with a motor, the clamp arm to maintain a magnitude (e.g., distance) of the gap between the ultrasonic blade and the clamp arm while driving the ultrasonic blade, as at step 410. For instance, while driving the ultrasonic blade 128 with the ultrasonic transducer 120, the controller 102 may continuously, or periodically, detect the gap between the clamp arm 140 and the ultrasonic blade 128 with the sensor 141. While driving the ultrasonic blade 128, the gap between the ultrasonic blade 128 and the clamp arm 140 may change due to the geometry of the tissue. For instance, when sealing tissue, the tissue captured between the clamp arm 140 and the ultrasonic blade 128 may deform (e.g., compress or expand), thereby causing the force that the tissue exerts on the clamp arm 140 to change (increase or decrease). If the controller 102 detects a change in the gap between the clamp 140 and the ultrasonic blade 128, the controller 102 may energize (actuate) the motor 121 to maintain the desired gap between the clamp arm 140 and the ultrasonic blade 128.

For instance, if the controller 102 detects the gap between the clamp 140 and the ultrasonic blade 128 increasing, such as due to the tissue expanding, the controller 102 may energize (actuate) the motor 121 to drive (rotate) the clamp arm 140 toward the ultrasonic blade 128 to reduce the gap, thereby maintaining the desired gap between the clamp arm 140 and the ultrasonic blade 128. Conversely, if the controller 102 detects the gap between the clamp 140 and the ultrasonic blade 128 decreasing, such as due to the tissue being compressed, the controller 102 may energize (actuate) the motor 121 to drive (rotate) the clamp arm 140 away from the ultrasonic blade 128 to increase the gap, thereby maintaining the desired gap between the clamp arm 140 and the ultrasonic blade 128.

A consistent, or substantially consistent, gap between the clamp arm 140 and ultrasonic blade 128 helps reduce the amount of pressure applied to the tissue grasped between the clamp arm 140 and the ultrasonic blade 128 while the ultrasonic blade 128 frictionally oscillates against the tissue. This may prove advantageous in helping to prevent, or at least substantially reduce the chance of, the ultrasonic blade 128 from cutting the tissue. Rather, a consistent, or substantially consistent, gap between the clamp arm 140 and ultrasonic blade 128 increases the chance that the ultrasonic blade 128 will only seal tissue, as opposed to cutting the tissue.

When the user input corresponds to the second (tissue cutting) mode, the method 400 may include sensing a gap between the ultrasonic blade and the clamp arm, as at step 412. For instance, based on the controller 102 receiving a user input indicative of the second (tissue cutting) mode of the surgical instrument 104, the controller 102 may sense, via the sensor 141, the gap (distance) between the clamp arm 140 and the ultrasonic blade 128.

The method 400 may further include driving the ultrasonic blade with an ultrasonic transducer, as at step 414. For instance, the controller 102 may energize (actuate) the ultrasonic transducer 120, such as with the ultrasonic generator 114 (FIG. 2), to oscillate (drive) the ultrasonic blade 128. The controller 102 may energize the ultrasonic transducer 120 based on a user providing an input to the controller 102, such as via the input device 110 or the footswitch 130 (FIG. 2), or based on a user providing an input to the instrument 104, such as via the toggle buttons 134a,b. In other embodiments, the controller 102 may energize (actuate) the ultrasonic transducer 120 automatically, such as based on the controller 102 detecting the tissue being captured between the clamp arm 140, 146 and the ultrasonic blade 128, 149. The controller 102 may detect the tissue being captured by applying a subtherapeutic energy signal to the electrode 147 and sensing (detecting) a change in impedance.

The method 400 may further include driving, with a motor, the clamp arm to reduce the magnitude of the gap between the ultrasonic blade and the clamp arm while driving the ultrasonic blade, as at step 416. For instance, while driving the ultrasonic blade 128 with the ultrasonic transducer 120, the controller 102 may continuously, or periodically, energize (actuate) the motor to change (reduce) the gap between the clamp arm 140 and the ultrasonic blade 128, thereby increasing, or at least substantially maintaining, the amount of pressure applied to the tissue grasped between the clamp arm 140 and ultrasonic blade 128. The controller 102 may energize (actuate) the motor to change (reduce) the gap between the clamp arm 140 and the ultrasonic blade 128 at a constant, or substantially constant, rate (i.e., a linearly rate of change), at a non-constant, or substantially non-constant, rate (i.e., a non-linearly rate of change), or in a stepwise fashion (i.e., energize the motor to rotate the clamp arm 140, de-energize the motor to pause rotation, and repeat), or combinations thereof.

Increasing, or maintaining, the pressure applied to the tissue may increase the amount of energy that the ultrasonic blade 128 delivers to the tissue, which allows, or at least substantially increases the chances of, the ultrasonic blade 128 to cut the tissue, rather than just sealing the tissue.

Accordingly, the foregoing systems and methods enable a user to selectively control whether an ultrasonic surgical instrument, such as the ultrasonic surgical instrument 104 or the multifunction surgical instrument 108, cuts or seals tissue.

While the foregoing principles were described with respect to handheld surgical instruments (e.g., surgical instruments 104 and 108; FIG. 1), the principles are equally applicable to robotic surgical systems and instruments, examples of which are described in U.S. Pat. No. 11,424,027, titled “METHOD FOR OPERATING SURGICAL INSTRUMENT SYSTEMS”, which issued on Aug. 23, 2022, which is hereby incorporated by reference in its entirety herein.

### Embodiments Disclosed Herein Include

A. A surgical system comprising a surgical instrument comprising an ultrasonic drive system that includes an ultrasonic blade and an ultrasonic transducer energizable to drive the ultrasonic blade, a temperature sensor to sense a temperature of the ultrasonic transducer, and a controller in operable communication with the ultrasonic transducer and the temperature sensor, the controller being operable to receive a user input, set a temperature range of the ultrasonic blade based on the user input, drive, with the ultrasonic transducer, the ultrasonic blade a first manner, sense, with the temperature sensor, the temperature of the ultrasonic transducer, determine the natural frequency of the ultrasonic drive system, determine a temperature of the ultrasonic blade based on the natural frequency and the temperature of the ultrasonic transducer, compare the temperature of the ultrasonic blade to the temperature range, and drive, with the ultrasonic transducer, the ultrasonic blade in a second manner different than the first manner based on the comparison.

B. A surgical system comprising a surgical instrument comprising an ultrasonic drive system that includes an ultrasonic blade and an ultrasonic transducer energizable to drive the ultrasonic blade, a temperature sensor to sense a temperature of the ultrasonic transducer, and a controller in operable communication with the ultrasonic transducer and the temperature sensor, the controller being operable to receive a user input, set a temperature set point of the ultrasonic blade based on the user input, drive, with the ultrasonic transducer, the ultrasonic blade a first manner, sense, with the temperature sensor, the temperature of the ultrasonic transducer, determine the natural frequency of the ultrasonic drive system, determine a temperature of the ultrasonic blade based on the natural frequency and the temperature of the ultrasonic transducer, compare the temperature of the ultrasonic blade to the temperature set point, and drive, with the ultrasonic transducer, the ultrasonic blade in a second manner different than the first manner based on the comparison.

C. A surgical system comprising an ultrasonic drive system that includes an ultrasonic blade and an ultrasonic transducer energizable to drive the ultrasonic blade, a temperature sensor to sense a temperature of the ultrasonic transducer, and a controller in operable communication with the ultrasonic transducer and the temperature sensor, the controller being operable to drive, with the ultrasonic transducer, the ultrasonic blade a first manner, sense, with the temperature sensor, the temperature of the ultrasonic transducer, estimate a temperature of the ultrasonic blade based on a natural frequency of the ultrasonic drive system and the temperature of the ultrasonic transducer, compare the temperature of the ultrasonic blade to a temperature range, and drive, with the ultrasonic transducer, the ultrasonic blade in a second manner different than the first manner based on the comparison.

Each of the embodiments A, B and C may have one or more of the following additional elements in any combination: Element 1: wherein the controller drives the ultrasonic blade in the second manner when the temperature of the ultrasonic blade is outside of the temperature range. Element 2: wherein to drive the ultrasonic blade in the first manner, the controller vibrates the ultrasonic blade within a first range of motion, and wherein to drive the ultrasonic blade in the second manner, the controller vibrates the ultrasonic blade within a second range of motion different than the first range of motion. Element 3: wherein to drive the ultrasonic blade in the first manner, the controller vibrates the ultrasonic blade at a first frequency, and wherein to drive the ultrasonic blade in the second manner, the controller vibrates the ultrasonic blade at a second frequency different than the first frequency. Element 4: wherein the temperature sensor is coupled to the ultrasonic transducer. Element 5: wherein the user input is indicative of an operating mode of the surgical instrument. Element 6: wherein the operating mode comprises a cutting mode or a sealing mode. Element 7: wherein, in the sealing mode, the temperature range of the ultrasonic blade is about 145° C. to about 155° C. Element 8: wherein, in the sealing mode, the temperature range of the ultrasonic blade is about 270° C. to about 280° C. Element 9: wherein the controller is operable to drive the ultrasonic blade in the second manner when the temperature of the ultrasonic blade is different than the temperature set point. Element 10: wherein to drive the ultrasonic blade in the first manner, the controller vibrates the ultrasonic blade within a first range of motion, and wherein to drive the ultrasonic blade in the second manner, the controller vibrates the ultrasonic blade within a second range of motion different than the first range of motion. Element 11: wherein to drive the ultrasonic blade in the first manner, the controller vibrates the ultrasonic blade at a first frequency, and wherein to drive the ultrasonic blade in the second manner, the controller vibrates the ultrasonic blade at a second frequency different than the first frequency. Element 12: wherein the temperature sensor is coupled to the ultrasonic transducer. Element 13: wherein the user input is indicative of an operating mode of the surgical instrument. Element 14: wherein the operating mode comprises a cutting mode or a sealing mode. Element 15: wherein, in the sealing mode, the temperature set point of the ultrasonic blade is about 150° C. Element 16: wherein, in the sealing mode, the temperature set point of the ultrasonic blade is about 275° C. Element 17: wherein the controller is operable to drive the ultrasonic blade in the second manner based on the estimated temperature of the ultrasonic blade being outside of the temperature range.

By way of non-limiting example, exemplary combinations applicable to A, B and C include: Element 6 and Element 7; Element 6 and Element 7; two more of Elements 1 through 8; Element 14 and Element 15; Element 14 and Element 16; two or more of Elements 9 through 16.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A surgical system, comprising:
a surgical instrument comprising an ultrasonic drive system that includes an ultrasonic blade and an ultrasonic transducer energizable to drive the ultrasonic blade;
a temperature sensor to sense a temperature of the ultrasonic transducer; and
a controller in operable communication with the ultrasonic transducer and the temperature sensor, the controller being operable to:
receive a user input;
set a temperature range of the ultrasonic blade based on the user input;
drive, with the ultrasonic transducer, the ultrasonic blade in a first manner;
sense, with the temperature sensor, the temperature of the ultrasonic transducer;
determine the natural frequency of the ultrasonic drive system;
determine a temperature of the ultrasonic blade based on the natural frequency and the temperature of the ultrasonic transducer;
compare the temperature of the ultrasonic blade to the temperature range; and
drive, with the ultrasonic transducer, the ultrasonic blade in a second manner different than the first manner based on the comparison.

2. The surgical system of claim 1, wherein the controller drives the ultrasonic blade in the second manner when the temperature of the ultrasonic blade is outside of the temperature range.

3. The surgical system of claim 1, wherein to drive the ultrasonic blade in the first manner, the controller vibrates the ultrasonic blade within a first range of motion, and wherein to drive the ultrasonic blade in the second manner, the controller vibrates the ultrasonic blade within a second range of motion different than the first range of motion.

4. The surgical system of claim 1, wherein to drive the ultrasonic blade in the first manner, the controller vibrates the ultrasonic blade at a first frequency, and wherein to drive the ultrasonic blade in the second manner, the controller vibrates the ultrasonic blade at a second frequency different than the first frequency.

5. The surgical system of claim 1, wherein the temperature sensor is coupled to the ultrasonic transducer.

6. The surgical system of claim 1, wherein the user input is indicative of an operating mode of the surgical instrument.

7. The surgical system of claim 6, wherein the operating mode comprises a cutting mode or a sealing mode.

8. The surgical system of claim 7, wherein, in the sealing mode, the temperature range of the ultrasonic blade is about 145° C. to about 155° C.

9. The surgical system of claim 7, wherein, in the sealing mode, the temperature range of the ultrasonic blade is about 270° C. to about 280° C.

10. A surgical system, comprising:
a surgical instrument comprising an ultrasonic drive system that includes an ultrasonic blade and an ultrasonic transducer energizable to drive the ultrasonic blade;
a temperature sensor to sense a temperature of the ultrasonic transducer; and
a controller in operable communication with the ultrasonic transducer and the temperature sensor, the controller being operable to:
receive a user input;
set a temperature set point of the ultrasonic blade based on the user input;
drive, with the ultrasonic transducer, the ultrasonic blade in a first manner;
sense, with the temperature sensor, the temperature of the ultrasonic transducer;
determine the natural frequency of the ultrasonic drive system;
determine a temperature of the ultrasonic blade based on the natural frequency and the temperature of the ultrasonic transducer;
compare the temperature of the ultrasonic blade to the temperature set point; and
drive, with the ultrasonic transducer, the ultrasonic blade in a second manner different than the first manner based on the comparison.

11. The surgical system of claim 10, wherein the controller is operable to drive the ultrasonic blade in the second manner when the temperature of the ultrasonic blade is different than the temperature set point.

12. The surgical system of claim 10, wherein to drive the ultrasonic blade in the first manner, the controller vibrates the ultrasonic blade within a first range of motion, and wherein to drive the ultrasonic blade in the second manner, the controller vibrates the ultrasonic blade within a second range of motion different than the first range of motion.

13. The surgical system of claim 10, wherein to drive the ultrasonic blade in the first manner, the controller vibrates the ultrasonic blade at a first frequency, and wherein to drive the ultrasonic blade in the second manner, the controller vibrates the ultrasonic blade at a second frequency different than the first frequency.

14. The surgical system of claim 10, wherein the temperature sensor is coupled to the ultrasonic transducer.

15. The surgical system of claim 10, wherein the user input is indicative of an operating mode of the surgical instrument.

16. The surgical system of claim 15, wherein the operating mode comprises a cutting mode or a sealing mode.

17. The surgical system of claim 16, wherein, in the sealing mode, the temperature set point of the ultrasonic blade is about 150° C.

18. The surgical system of claim 16, wherein, in the sealing mode, the temperature set point of the ultrasonic blade is about 275° C.

19. A surgical system, comprising:
an ultrasonic drive system that includes an ultrasonic blade and an ultrasonic transducer energizable to drive the ultrasonic blade;
a temperature sensor to sense a temperature of the ultrasonic transducer; and
a controller in operable communication with the ultrasonic transducer and the temperature sensor, the controller being operable to:
drive, with the ultrasonic transducer, the ultrasonic blade in a first manner;
sense, with the temperature sensor, the temperature of the ultrasonic transducer;
estimate a temperature of the ultrasonic blade based on a natural frequency of the ultrasonic drive system and the temperature of the ultrasonic transducer;
compare the temperature of the ultrasonic blade to a temperature range; and
drive, with the ultrasonic transducer, the ultrasonic blade in a second manner different than the first manner based on the comparison.

20. The surgical system of claim 19, wherein the controller is operable to drive the ultrasonic blade in the second manner based on the estimated temperature of the ultrasonic blade being outside of the temperature range.
